(19) United States
(12) Patent Application Publication
(10) Pub. No.: US 2025/0318850 A1
(43) Pub. Date: Oct. 16, 2025

(54) MEDICAL SYSTEM AND METHOD FOR OPERATING A MEDICAL SYSTEM FOR DETERMINING THE POSITION OF AN ACCESS DEVICE

(71) Applicant: KARL STORZ SE & Co. KG, Tuttlingen (DE)

(72) Inventor: Jochen STEFAN, Tuttlingen (DE)

(73) Assignee: KARL STORZ SE & Co. KG, Tuttlingen (DE)

(21) Appl. No.: 18/881,604

(22) PCT Filed: Jul. 6, 2023
(86) PCT No.: PCT/EP2023/068679

§ 371 Date: Jan. 6, 2025

(30) Foreign Application Priority Data
Jul. 7, 2022 (DE) 10 2022 117 010.1

(51) Int. Cl. A61B 17/34; A61B 17/00

(52) U.S. Cl. CPC A61B 17/3403; A61B 2017/00039; A61B 2017/00057; A61B 2017/00075; A61B 2017/00212; A61B 2017/3409

(57) ABSTRACT

A medical system for operating an assisted system for minimally invasive surgery, includes a robot configured to hold and move a medical instrument; an access device arrangeable to provide access to a body cavity for a medical instrument held by the robot, wherein the access device includes a position indicator; a measuring unit having a sensor configured to carry out a sensor measurement directed at the position indicator; a position determining unit configured to determine a position of the access device according to the sensor measurement; a pivot point determining unit configured to determine a pivot point for the access device according to the determined position of the access device, and a robot controller configured to determine control commands for controlling the robot according to the determined pivot point, in such a way that the robot performs pivot movements of the medical instrument about the pivot point.

### CROSS-REFERENCE TO RELATED APPLICATIONS

This application is the U.S. national stage of PCT/EP2023/068679 filed on Jul. 6, 2023, which claims priority of German Patent Application No. DE 10 2022 117 010.1 filed on Jul. 7, 2022, the contents of which are incorporated herein.

### TECHNICAL FIELD

The present disclosure relates to a medical system, in particular a system-assisted system for minimally invasive surgery, for determining the position of an access device, and to a method for operating a medical system, in particular a system-assisted system for minimally invasive surgery, for determining the position of an access device.

### BACKGROUND

Well-known examples of such systems are the Da Vinci surgical systems from Intuitive Surgical, Inc. Such medical systems can be used to support minimally invasive procedures such as urological procedures, general laparoscopic procedures, general thoracoscopic procedures, thoracoscopic-assisted cardiac procedures, and/or transoral otolaryngologic procedures. In order to carry out a procedure inside a patient's body cavity, a user controls medical instruments, in particular endoscopic instruments, at a control console via control commands such as hand movements. A robot moves the medical instruments in a manner corresponding to the control commands. Access devices, such as trocars, are used to provide access to the body cavity. For this purpose, the access devices are guided through a layer of tissue covering the corresponding body cavity at passage points, wherein the passage point is selected to be as small as possible in order to reduce injury to the patient. The medical instruments can be fed through the lumen of the access devices at the passage points of the patient's body cavity. The user can then carry out a minimally invasive procedure via control commands, wherein a robot implements control commands, predefined by the user, to control medical instruments.

During a procedure, it may be necessary to pivot the medical instruments about a pivot point. In order to minimize stretching and possible injury to tissue in the region of the passage points, the medical instruments should be pivoted about a pivot point located within the tissue and the access device at the particular passage points. For example, if the body cavity is the abdominal cavity, the pivot point of a medical instrument should be located within the abdominal wall and at the passage point of an access device through the abdominal wall. If the medical instrument is pivoted about a deviating pivot point, serious injuries to the patient may occur—for example, because the medical instrument moves sideways and exerts forces on the tissue starting from the passage point.

According to the prior art, a robot arm of the robot is mechanically coupled to the access device by means of a mechanical coupling unit. As a result, on the one hand, relative movements between the robot arm and the access device are prevented, and, on the other, since the position of the robot arm is known, the position of the access device is also known. A marking can be provided on the access device for identifying the pivot point, also known as the swivel point, which must be arranged in a suitable spatial relationship to the punctured tissue when positioning the access device in order to position the pivot point correctly. A medical instrument provided on the robotic arm and fed at least partially to the body cavity via the access device is pivoted by the robot about the pivot point. With this solution, the size, volume, and/or space requirements of the mechanical coupling unit in particular are considerable.

### SUMMARY

Based upon the prior art, it is an object of the present disclosure to achieve a high degree of freedom and security when using an access device.

This object is achieved according to the disclosure by a medical system with the features of claim 1, a method with the features of claim 15, program code with the features of claim 16, and a computer-readable medium with the features of claim 17.

The present disclosure provides a medical system, in particular a system-assisted system for minimally invasive surgery.

The medical system comprises a robot configured to hold and move a medical instrument, an access device arrangeable to provide access to a body cavity for a medical instrument held by the robot, wherein the access device comprises at least one position indicator, a measuring unit having at least one sensor configured to carry out a sensor measurement directed at the at least one position indicator, a position determining unit configured to determine a position of the access device according to the sensor measurement, a pivot point determining unit configured to determine a pivot point for the access device according to the determined position of the access device, and a robot controller configured to determine control commands for controlling the robot according to the determined pivot point in such a way that the robot carries out pivot movements of the medical instrument about the pivot point.

Furthermore, a method for operating a medical system, in particular a system-assisted system for minimally invasive surgery, is provided. The medical system comprises a robot configured to hold and move a medical instrument, and an access device arrangeable to provide access to a body cavity for a medical instrument held by the robot, wherein the access device comprises at least one position indicator. The method according to the disclosure comprises the following steps:
carrying out a sensor measurement directed at the at least one position indicator;
determining the position of the access device according to the sensor measurement;
determining a pivot point for the access device according to the determined position of the access device; and
generating control commands for controlling the robot according to the determined pivot point in such a way that the robot carries out pivot movements of the medical instrument about the pivot point.

According to the disclosure, pivot movements of the medical instrument about the pivot point of the access device can thus be carried out by means of the robot, wherein the positions of the access device and the pivot point are determined by means of at least one sensor and units configured for this purpose. This can result in a significant reduction in the size, volume, and/or space requirements of the mechanical structure of the robot. This in turn has several advantages. For example, by reducing the size, volume, and/or space requirements of the mechanical structure of the robot, the moving and/or accelerated weight of robot arms of the robot can be reduced, and thus dynamic movements of the robot arm can be achieved, in particular with, for example, higher bandwidth, higher precision, and/or higher accuracy. In addition, the lower moving and/or accelerated weight results in lower energy requirements for operating the robot. Furthermore, it is advantageous to reduce the space required by medical systems in the immediate vicinity, e.g., within half a meter, of a patient on whom a procedure is being performed. As a result, for example, a larger number of medical systems and/or medical instruments can be provided for carrying out the procedure on the patient. It also facilitates access for the treating persons. In addition, coupling and/or mutual interference between different medical systems and/or medical instruments can be reduced, and/or, for example, the freedom of movement of individual medical systems and/or medical instruments can be increased.

For example, the robot can be part of a medical system configured to perform a procedure on a patient. In particular, the robot can be part of a robot-assisted system for minimally invasive surgery. The robot can comprise a plurality of robot arms, wherein each robot arm can be configured to hold and move at least one medical instrument. In particular, the robot can be configured to hold and move the medical instrument in such a way that it is positioned within a patient's body cavity with a high degree of precision. Positioning can be carried out using control commands that are predefined by a user via a control console. Positioning within a patient's body cavity can comprise a pivot movement of at least one medical instrument. The robot can be configured to pivot the at least one medical instrument about any desired pivot point, wherein the position of the pivot point can be at least substantially unchanged over the entire period of a procedure.

Medical instruments that can be provided at least partially on a robot can comprise any medical instruments usable in connection with systems for minimally invasive surgery. endoscopy, and/or minimally invasive diagnostics. For example, the medical instrument can comprise an endoscope, a coagulation instrument, an end effector, an exoscope, forceps, scissors, and/or the like and/or at least a part thereof. Information about the position of medical instruments and/or access devices assigned to a robot arm can be used in a useful manner for controlling further robot arms according to the disclosure. For example, the spatial relationship between a plurality of access devices can be known, and therefore the robot can move and/or pivot further medical instruments according to the position of one of these access devices.

The access device can be a puncture instrument—for example, a trocar. The access device can alternatively or additionally comprise a device by means of which an access lumen is provided, but which is not itself configured for a puncture. By means of the access device. access to a patient's body cavity can be provided in order to carry out, for example, a surgical procedure and/or a diagnostic action. Body cavities can, for example, be the cranial cavity, the thoracic cavity, the abdominal cavity, and/or the pelvic cavity, in particular of a human patient. Typically, the access device passes through tissue and/or the like in order to provide access to a patient's body cavity.

At least one marking, e.g., a line and/or a dot, can be provided on the access device to indicate a preferred passage region of the access device. It can be provided that the access device be arranged on the patient in such a way that the preferred passage region is at least partially located within the penetrated tissue and/or the like. For example, if it is provided that the access device allow access to the abdominal cavity, it may be advantageous to arrange the at least one marking, which indicates a preferred passage region of the access device, in such a way that it is located in the region in which the abdominal wall is penetrated by the access device. The preferred access region can define at least one target pivot point. The target pivot point can be a pivot point and/or a trocar point. The at least one marking can indicate the target pivot point. The access device can comprise a tube designed to make possible a feed of a medical instrument through the tube into the body cavity.

The at least one position indicator can be arranged at a proximal end and/or in a proximal half and/or in a proximal third of the access device. In particular, the at least one position indicator can be arranged proximally with respect to the target pivot point and/or with respect to the passage region. In some embodiments, the at least one position indicator is arranged in such a way that it is located outside the body cavity in a state in which the access device establishes access to a body cavity as intended. In other embodiments, the at least one position indicator and/or at least one further position indicator can be arranged in a distal half and/or in a distal third and/or at a distal end of the access device—for example, in such a way that it is located within the body cavity in a state in which the access device establishes access to a body cavity as intended.

The at least one position indicator can be active or passive. The at least one position indicator can be configured to make it possible to determine the position of the access device—for example, on the basis of a distance measurement and/or triangulation.

The measuring unit can be configured to provide information with respect to the position of the at least one position indicator. This information can be based upon the sensor measurement. In some embodiments, providing the information does not comprise analyzing the position of parts of the robot—for example, information about the position, angular position, control, and/or performance of actuators of parts of the robot. In some cases according to the disclosure, the position can be determined and/or determinable directly and/or immediately from an analog and/or digital signal of the measuring unit.

The position of the access device determined by the position determining unit can comprise the position and/or orientation of the access device. In particular, the position determining unit can process information about the position of at least three, preferably three or four, position indicators, in order to determine the position of the access device. In particular, the position determining unit can be configured to determine the position of the access device in a robot coordinate system. The position of the access device can be determined at least partially relative to the robot.

The pivot point determining unit can determine the pivot point, for example, using a geometric relationship, in particular using a distance, between the at least one position indicator and any, preferably selectable, spatial point. The determination of the pivot point can preferably be provided in the robot coordinate system. For example, the distance can be a distance to an origin of the robot coordinate system and/or to any point defined in the robot coordinate system.

The determined pivot point can be provided as a pivot point for pivot movements of the robot. In particular, the robot can be controlled in such a way that medical instruments provided on the robot are at least partially pivoted about the pivot point. The pivot point can be substantially unchangeable. The pivot point can substantially coincide with the target pivot point.

The measuring unit and/or the position determining unit and/or the pivot point determining unit can, in particular apart from sensor system, be at least partially or completely integral—for example, in a common control unit and/or a common computing unit.

Advantageously, the access device can be free of a connection, in particular a mechanical connection, to the robot. In other words, the pivot point is defined without contact. In particular, “free of a connection to the robot” can be understood to mean that no coupling unit is provided that couples the access device to the robot in a fixed position. In this respect, even in a state in which the robot transmits forces to the access device via the medical instrument, the access device can be free of a connection to the robot. This is the case in some embodiments of the disclosure. Pivot movements can be transferred from the robot to the access device via the medical instrument inserted into the access device, wherein the access device is otherwise not connected to the robot and/or coupled to it. If the medical instrument is pivoted about the pivot point, this causes a pivot movement of the access device about the pivot point. for example. In this way, the access device can be pivoted about a controlled and substantially unchangeable pivot point as required, even though it is not coupled to the robot. This is particularly advantageous, since no mechanical connection and/or connection unit between the robot and the access device needs to be provided. A medical system according to the disclosure thus has a smaller spatial extent, space requirement, size, weight, and/or the like than a medical system from the prior art.

In advantageous embodiments, the measuring unit can be configured to measure a distance to the at least one position indicator. For example, a geometric positional relationship between the at least one position indicator and at least one part of the measuring unit, in particular the at least one sensor, can be determined. Advantageously, the position of at least one part of the measuring unit, in particular of at least one sensor, in the robot coordinate system is known. Consequently, the position of the at least one position indicator can be determined in the robot coordinate system.

Furthermore, the at least one position indicator can comprise a signal generator configured to generate an electromagnetic signal. The electromagnetic signal can be a radio signal. The electromagnetic signal can contain information with respect to the position indicator—for example, an identification. This is particularly advantageous if there are obstacles, such as objects, people, and/or the like, between the measuring unit and the at least one position indicator.

Furthermore, the access device can comprise an energy storage unit configured to supply the at least one position indicator with energy. Such an energy storage unit can comprise a battery, for example. The energy storage unit can be rechargeable. In some embodiments, the access device can comprise a receiver configured to receive energy for charging the energy storage unit as in a wireless energy transfer—for example, an inductive coupling and/or capacitive coupling receiver. Alternatively or additionally, the energy storage unit can be removed and/or connected without tools.

According to some embodiments, the sensor of the measuring unit can be an electromagnetic detection sensor. This is particularly advantageous if at least one position indicator comprises a signal generator configured to generate an electromagnetic signal. In particular, one corresponding embodiment makes possible a sensor measurement and/or position determination despite obstacles between the measuring unit and the at least one position indicator. An electromagnetic sensor measurement and/or position determination can, for example, be carried out more precisely, more accurately, and/or more quickly, i.e., with a higher repetition rate, than, for example, an optical sensor measurement and/or position determination. In addition, the identification and/or differentiation of various position indicators can be carried out easily and reliably using the electromagnetic signal.

In some embodiments, the medical system can comprise at least one position indicator comprising at least one optically detectable marking. The optically detectable marking can comprise at least one geometric shape and/or at least one character and/or text and/or a code such as a QR code or a bar code and/or at least one color. If a plurality of position indicators are provided, these can comprise different and/or distinguishable, optically detectable markings that, for example, allow the identification of the relevant position indicator.

Furthermore, the sensor of the measuring unit can be an optical detection sensor. An optical detection sensor can be configured to detect the position of position indicators, which comprises at least one optically detectable marking. Furthermore, by means of the detection sensor, the marking can be detected, and thus the relevant position indicator can be identified. The position can be detected in such a way that a distance between the optical detection sensor and the position indicator is ascertained, in particular by means of triangulation. The position can also be detected in the robot coordinate system.

Advantageously, the access device can be free of an energy storage unit. In particular, optical detection sensors can be free of an energy storage unit. Access devices that are stored free of an energy storage unit can require less maintenance and/or be designed for longer operation without maintenance. In addition, the lack of an energy storage unit can simplify autoclaving.

Furthermore, the measuring unit can be configured to carry out an image detection of the at least one position indicator. For example, an optical detection sensor can comprise at least one camera. The image detection of the at least one position indicator is characterized in particular by a high degree of reliability and a low error rate. The optical detection sensor can comprise a stereo camera. Furthermore, at least two optical detection sensors can be provided. which together make stereoscopic image detection possible.

In some embodiments, the access device can comprise a plurality of position indicators arranged at different positions of the access device. The position determining unit can be configured to take into account the different positions of the position indicators when determining the position of the access device. In particular, it can be advantageous to arrange at least three position indicators with a known geometric relationship to one another on the access device. Alternatively or additionally, a geometric relationship between the position indicators and the marking indicating the passage region and/or the target pivot point can be known. The position determining unit and/or the pivot point determining unit can be configured to process any of the geometric relationships mentioned. These can be predefinable and/or predefined. If the distance of each of the at least three position indicators to a reference point is now determined, the position of the access device, e.g., comprising the distance to the reference point and the orientation in a coordinate system, preferably the robot coordinate system, can be determined by, for example, triangulation. Furthermore, it can be advantageous to provide redundant position indicators on the access device in order to increase the reliability of the position determination.

Furthermore, the robot controller can be configured to generate control commands for controlling the robot with respect to a robot coordinate system, wherein the pivot point determining unit can be configured in order to determine the pivot point in relation to the robot coordinate system. A simple calculation of control commands for the robot can then be made possible in particular if the pivot point is determined in the robot coordinate system. A single coordinate system, preferably the robot coordinate system, can reduce the computational effort and/or the susceptibility to errors when generating control commands for controlling the robot.

According to some embodiments, the access device can have a predefined and/or predefinable target pivot point, wherein it is provided that the target pivot point substantially coincide with the pivot point during a performance of the pivot movements of the medical instrument, and wherein the pivot point determining unit can be arranged to take into account a geometric relationship between the position indicator and the target pivot point when determining the pivot point for the access device. In particular, the access device can comprise standardized geometric dimensions, so that at least the position indicator arranged on the access device is at a known distance from the target pivot point. For example, a user can select an access device intended for a procedure from a list of several different access devices for which the mentioned standardization is provided. Alternatively or additionally, a plurality of predefinable target pivot points can be arranged on the access device. A target pivot point of an access device can be identified by at least one marking. Here, this can be the marking that indicates the passage region. A marking in the case of a plurality of predefinable target pivot points can comprise a scale, for example. Advantageously, the pivot point is determined by the pivot point determining unit, taking into account geometric positional relationships between at least one position indicator and the target pivot point, in such a way that the pivot point substantially coincides with the target pivot point. In such a case, the pivot point can be determined in such a way that it is located in the passage region. Consequently, if the robot is controlled by the robot controller in such a way that the robot carries out pivot movements of the medical instrument about the pivot point, the medical instrument is substantially pivoted about the region of the tissue penetrated by the access device. As a result, injuries to the penetrated tissue are advantageously prevented. If, on the other hand, the medical instrument were to be pivoted about a point other than the target pivot point, i.e., not about the region of the penetrated tissue, and/or additionally moved laterally to a considerable extent, this could result in massive injuries to the patient during a procedure.

Furthermore, the medical system can comprise a user interface via which a user can predefine the target pivot point and, in particular, a distance of the target pivot point from the at least one position indicator. For example, it may be advantageous for the user to obtain knowledge of the geometric positional relationship between at least one position indicator and a target pivot point, which is advantageously arranged in the region of the penetrated tissue, and to predefine it to the medical system via the user interface for determining the pivot point. For example, the user can predetermine where he wants to set the target center of gravity. From this information and a known geometry of the access device, including a known position of the at least one position indicator or known positions of a plurality of position indicators, the distance of the target pivot point from the at least one position indicator can then be determined—for example, by the position determining unit and/or the pivot point determining unit. This is particularly advantageous since, as a result, variable pivot points can be set, and, for example, relative movements between the access device and the patient can be reacted to.

Furthermore, a program code can be provided according to the disclosure, comprising instructions that, when executed by a processor, cause the method according to the disclosure to be carried out.

Furthermore, a computer program product can be provided, comprising a machine-readable medium on which the program code is stored.

The disclosure is explained below using figures by way of example. The drawings, the description, and the claims contain numerous features in combination. A person skilled in the art will also, expediently, consider the features individually and use them in combination as appropriate in the context of the claims.

### BRIEF DESCRIPTION OF DRAWINGS

If there is more than one example of a particular object, only one of them may be provided with a reference sign in the figures and in the description. The description of this example can be transferred accordingly to the other examples of the object. If objects are named using numerical words, such as first, second, third object, etc., these are used to name and/or assign objects. Accordingly, for example, a first object and a third object may be included, but not a second object. However, based upon numerical words, a number and/or sequence of objects could also be derived. The drawings show an embodiment of the disclosure. The drawings, the description and the claims contain numerous features in combination. A person skilled in the art will also, expediently, consider the features individually and combine them into useful further combinations.

FIG. 1 a schematic perspectival view of a medical system during a procedure on a patient;

FIG. 2 a schematic perspectival view of an access device of the system;

FIG. 3 a schematic perspectival view of an alternative embodiment of an access device;

FIG. 4 a schematic perspectival view of an access device arranged on the patient;

FIG. 5 a schematic perspectival view of the access device through which a medical instrument is guided; and

FIG. 6 a schematic flow diagram of a method for operating a medical system.

### DETAILED DESCRIPTION OF PREFERRED EMBODIMENTS

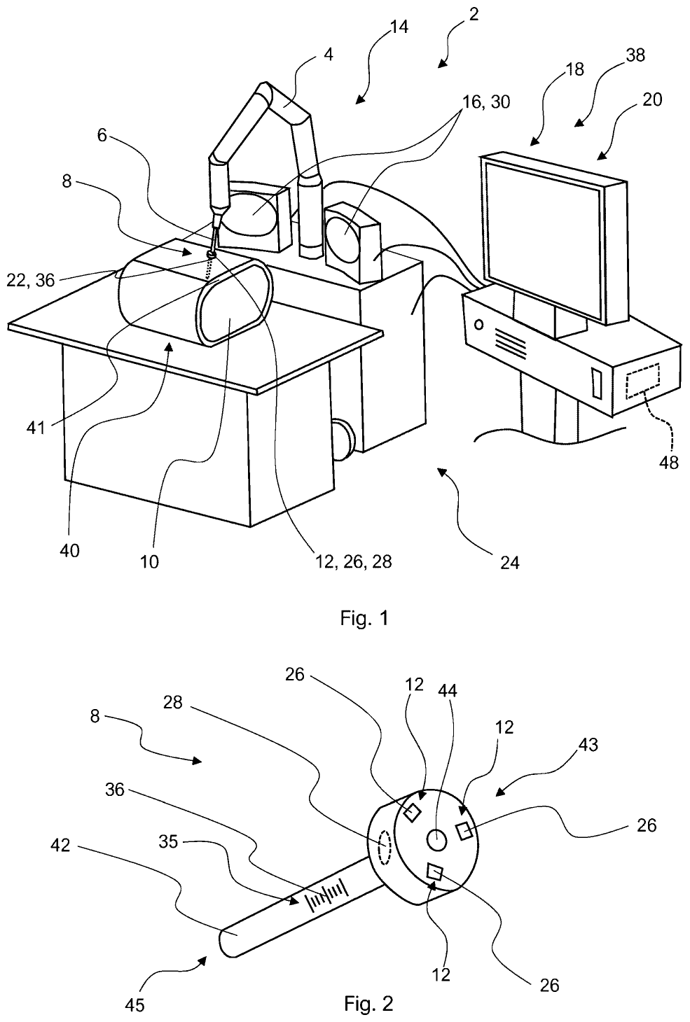

FIG. 1 shows by way of example a medical system 2 during a procedure on a patient 40. In the case shown, the medical system 2 is a system-assisted system for minimally invasive surgery. For reasons of clarity, only the part of the patient 40 on which the procedure is performed is shown schematically. According to this exemplary embodiment, the procedure is performed within a body cavity 10 of the patient 40. By way of example, the body cavity 10 is an abdominal cavity. The access device 8 is arranged on the patient 40 in order to provide access to the body cavity 10 for a medical instrument 6 held by the robot 4. In the case shown, the medical instrument 6 is an endoscope. However, it is understood that it can be any medical, in particular surgical and/or minimally invasive, instrument. Even if, according to this exemplary embodiment, only one medical instrument 6 and only one robot 4, or only one robot arm, is provided, it is understood that a plurality of, in particular also different, medical instruments 6 and/or a plurality of robots or robot arms can be provided according to the disclosure.

FIG. 2 shows the access device 8 in detail. The access device 12 comprises three position indicators 12, wherein these have a known geometric positional relationship, in particular a known distance, to one another. The position indicators 12 each comprise a signal generator 26. Examples of signal generators 26 are known, for example, from the NAVIR electromagnetic product from Karl Storz SE & Co. KG. The signal generators 26 are each configured to generate an electromagnetic signal. The signal generators are fed by an energy storage unit 28. In the exemplary case shown, the electromagnetic signal is a radio signal that contains an identification of the particular position indicator 12. Using the electromagnetic signal and its signal strength, it is thus possible to ascertain which of the position indicators 12 is involved and at what distance it is located.

Furthermore, a target pivot point 36 is marked on the access device 8 by an optical marker 35. The optical marker 35 is designed as a scale that allows the selection of different target pivot points 36. The distance between the target pivot point 36 or a fixed point of the optical marker 35 and each of the position indicators 12 and/or signal generators 26 is known. The access device 8 further comprises a tube 42 comprising a tubular lumen 44. The tube 42 and the tubular lumen 44 are configured in such a way that they provide access to the body cavity 10 for a medical instrument 6. For insertion into the body cavity 10, the medical instrument 6 can be passed through the access device 8 from a proximal end 43 of the access device 8 to a distal end 45 of the access device. An outer diameter of the shaft of the medical instrument 6 corresponds at least substantially to an inner diameter of the tube 42.

Such an access device 8 is arranged in FIG. 1 in such a way that the target pivot point 36 lies within the abdominal wall 41. A medical instrument 6 is fed to the body cavity 10 through the tube 42 of the access device 8.

Furthermore, the medical instrument 6 is held by a robot 4. The robot is controlled by a robot controller 24. A user (not shown) can control the robot via control commands in order to perform a procedure within the body cavity 10. During the procedure, it may be necessary to pivot the medical instrument 6 about a pivot point 22—for example, to reach and/or image different points within the body cavity 10.

In order to largely avoid injury to the abdominal wall 41, it is sensible to select the pivot point 22 of the medical instrument 6 in such a way that it largely coincides with the target pivot point 36 of the access device 8. In order to accomplish this, the medical system 2 comprises a measuring unit 14 having at least one sensor 16. By way of example, two sensors 16 are provided, which are spaced apart from one another and are thus in different spatial relationships to the position indicators 12. For this purpose, according to this exemplary embodiment, the position of the three position indicators 12 is determined by means of a sensor measurement by the sensors 16 of the measuring unit 14. According to this exemplary embodiment, the sensors 16 are electromagnetic detection sensors 30. These detect a signal strength. In addition, these can detect information contained in the electromagnetic signals, as a result of which the different position indicators 12 can be identified. It is understood that a sensible combination of position indicators 12 and sensors 16 is selected.

The system 10 further comprises a position determining unit 18. The position determining unit 18 can determine the position of the access device 8 relative to the sensors 16 according to the position of the three position indicators 12 by means of triangulation. Since the position of the sensors 16 in a robot coordinate system is known, the position of the access device 18 in the robot coordinate system can also be ascertained.

Furthermore, the system 10 comprises a pivot point determining unit 20. By means of the pivot point determining unit 20, any pivot point 22 of the access device 8 can be determined, wherein the pivot point 22 is determined, for example, via a geometric positional relationship to the position indicators 12 arranged on the access device 8, via a user input. In the case shown, the system 10 comprises a user interface 38 for this purpose. The user input can comprise a desired geometric positional relationship and can be specified via the user interface 38. For example, the user input comprises the information as to which point on the scale of the optical marker 35 the target pivot point 36 is to be located. As a result, the geometric positional relationship between the position indicators 12 and the target pivot point 36 of the access device 8 can be entered and/or selected. In this case, the pivot point determining unit 20 determines the pivot point 22 in such a way that it largely corresponds to the target pivot point 36. As a result, the robot controller 24 is placed in the position of determining control commands for operating the robot 4 in such a way that the robot 4 carries out pivot movements of the medical instrument 6 about the target pivot point 36. It is understood that the pivot point 22 does not necessarily have to correspond to the target pivot point 36. There may be situations in which a pivot point 22 that deviates from the target pivot point 36 is advantageous. The determination of such an advantageous pivot point 22 is also within the meaning of the disclosure.

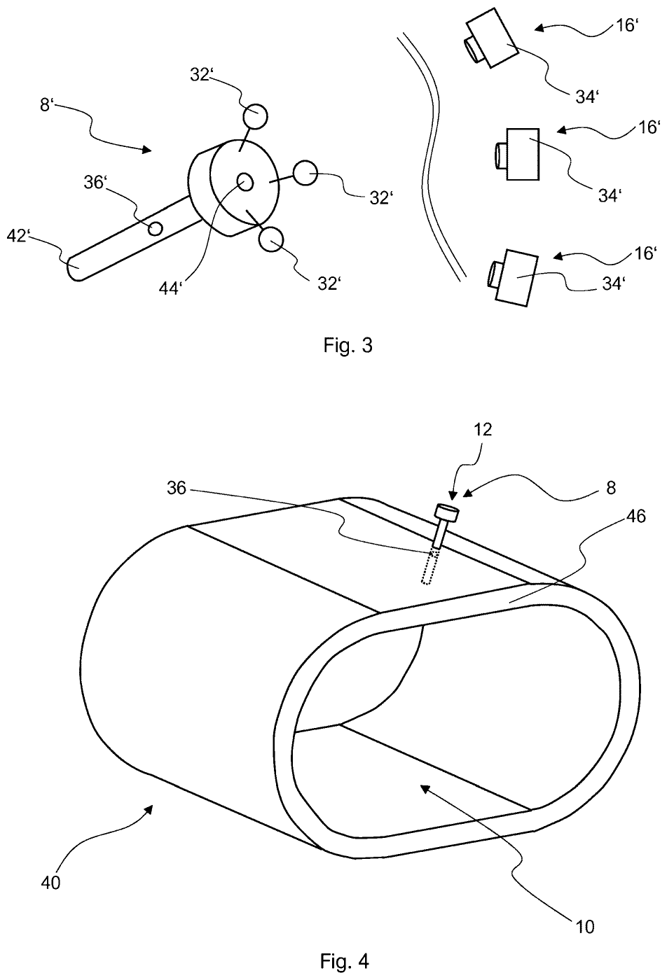

FIG. 3 shows an alternative embodiment of an access device 8′. Reference signs of this embodiment are provided with quotation marks to distinguish them. The alternative access device 8′ comprises position indicators 12′, which can be detected by means of an optical measurement. According to this embodiment, the position indicators 12 comprise optically detectable markings 32. Sensors 16′ of a measuring unit (not shown in full) are optical detection sensors 34′, e.g., cameras, which are configured to detect the position of the optically detectable markings 32′. Advantageously, at least two optical detection sensors 34′—according to this example, three—are provided. The introduction of redundancy is particularly advantageous when using optical detection sensors 34′. If at least a part of another unit, e.g., the robot 4 described above, the access device 8′, and/or a user or another object, is located between the optically detectable markings 32′ and one of the optical detection sensors 34′, such that this optical detection sensor 34′ cannot carry out a sensor measurement directed at the optically detectable markings 32′, at least temporarily, another one of the optical detection sensors 34′ can, however, still carry out a sensor measurement directed at the optically detectable markings 32′. Since at least three position indicators 12 are provided according to the exemplary embodiment in FIG. 3, the position determining unit 18 described above can determine the position of the access device 8′ by triangulation, for example. An access device 8′ according to FIG. 3 advantageously does not comprise an energy storage unit.

With respect to further features of the alternative access device 8′ and its use in a medical system and its design, reference is made to the above and the following description of the system 10.

FIG. 4 shows the access device 8 described above, which is arranged on a patient 40 to make possible a pivot movement about a target pivot point 36 within tissue layers 46 of the patient 40. The part of the access device 8 on which the position indicators 12 are provided is arranged outside the body cavity 10. A distal part of the access device extends into the body cavity 10 of the patient 40, wherein the access device 8 passes through the tissue layers 46. In order to make this passage possible, the access device 8 can be designed with a blade and/or sharp cutting edge, for example. Alternatively or additionally, the tissue layers 46 can be severed by a separately carried out cut and/or stitch and/or procedure, in order to provide a passage for the access device 8.

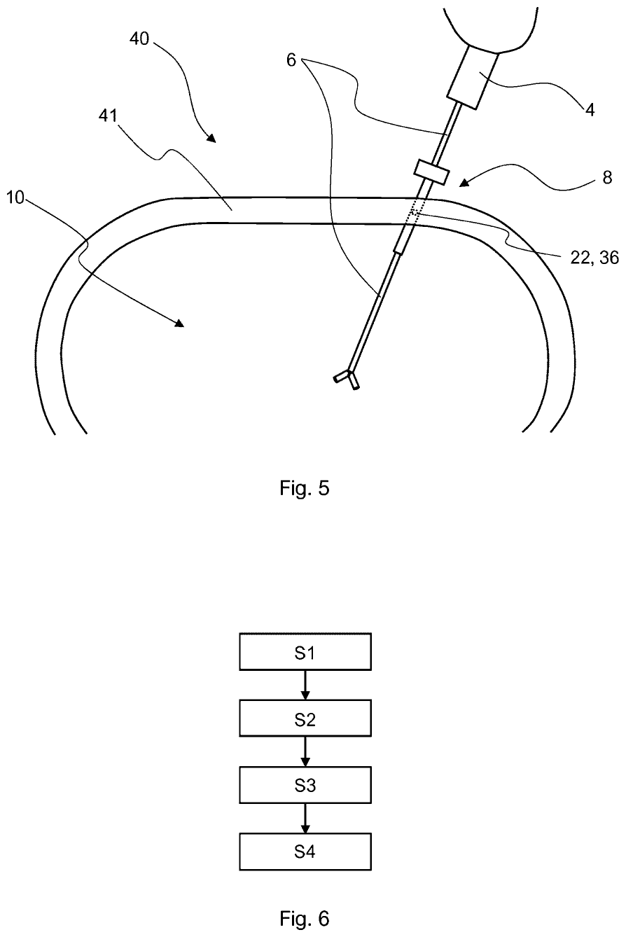

FIG. 5 shows the access device 8 arranged as shown in FIG. 4, wherein a medical instrument 6 is guided through the access device 8, so that it is partially located within the body cavity 10 of the patient 40. As mentioned, medical instrument 6 is an endoscope. The medical instrument 6 comprises, for example, a working lumen through which a tool such as a tissue forceps can be inserted into the body cavity 10 and observed. However, it is understood that any medical instrument 6 can be provided according to the disclosure.

The medical instrument 6 is arranged on a robot 4, which is only partially shown. The robot 4 is controlled by an user (not shown) via control commands, in order to carry out a procedure by means of the medical instrument 6 within the body cavity 10 of the patient 40. The user has configured the pivot point 22 of the robot 4 in such a way that it substantially corresponds to the target pivot point 36. If a pivot movement of the medical instrument 6 is now necessary during the procedure, the robot 4 pivots the medical instrument 6 substantially about the target pivot point 36.

FIG. 6 shows the steps of a method for operating a medical system 2. The method is explained by way of example with reference to the system 2 described above. The method comprises a step SI of carrying out a sensor measurement directed at the at least one position indicator 12. Furthermore, the method comprises a step S2 of determining a position of the access device 8 according to the sensor measurement. Furthermore, the method comprises a step S3 of determining a pivot point 22 for the access device 8 according to the determined position of the access device 8. In addition, the method comprises a step S4 of generating control commands for controlling the robot 4 according to the determined pivot point 22 in such a way that the robot 4 carries out pivot movements of the medical instrument 6 about the pivot point 22.

In the following, reference is once again made to FIG. 1. The system 10 comprises a computer program product 48 comprising a machine-readable medium—in this case, a memory. The computer program product 48 can be part of a control unit of the medical system 10. The machine-readable medium of the computer program product 48 stores program code comprising instructions that, when executed by a processor, cause the method to be carried out.

The drawings show an exemplary embodiment of the disclosure. The drawings, the description, and the claims contain numerous features in combination. A person skilled in the art will also, expediently, consider the features individually and combine them into useful further combinations. The disclosure relates to a medical system 2 and a method for operating a medical system 2, in particular a system-assisted system for minimally invasive surgery, comprising:
a robot 4 configured to hold and move a medical instrument 6;
an access device 8 arrangeable to provide access to a body cavity 10 for a medical instrument 6 held by the robot 4, wherein the access device 8 comprises at least one position indicator 12;
a measuring unit 14 having at least one sensor 16 configured to carry out a sensor measurement directed at the at least one position indicator 12;
a position determining unit 18 configured to determine a position of the access device 8 according to the sensor measurement;
a pivot point determining unit 20 configured to determine a pivot point 22 for the access device 8 according to the determined position of the access device 8, and
a robot controller 24 configured to determine control commands for controlling the robot 4 according to the determined pivot point 22 in such a way that the robot 4 carries out pivot movements of the medical instrument 6 about the pivot point 22.

1. A medical system, in particular a system-assisted system for minimally invasive surgery, comprising:
a robot configured to hold and move a medical instrument;
an access device arrangeable to provide access to a body cavity for a medical instrument held by the robot, wherein the access device comprises at least one position indicator;
a measuring unit having at least one sensor configured to carry out a sensor measurement directed at the at least one position indicator;
a position determining unit configured to determine a position of the access device according to the sensor measurement;
a pivot point determining unit configured to determine a pivot point for the access device according to the determined position of the access device; and
a robot controller configured to determine control commands for controlling the robot according to the determined pivot point in such a way that the robot carries out pivot movements of the medical instrument about the pivot point.

2. The medical system according to claim 1, wherein the access device is free of a connection to the robot.

3. The medical system according to claim 1, wherein the measuring unit is configured to measure a distance to the at least one position indicator.

4. The medical system according to claim 1, wherein the at least one position indicator comprises a signal generator configured to generate an electromagnetic signal.

5. The medical system according to claim 4, wherein the access device comprises an energy storage unit configured to supply the at least one position indicator with energy.

6. The medical system according to claim 1, wherein the sensor of the measuring unit is an electromagnetic detection sensor.

7. The medical system according to claim 1, wherein the at least one position indicator comprises at least one optically detectable marking.

8. The medical system according to claim 1, wherein the sensor of the measuring unit is an optical detection sensor.

9. The medical system according to claim 1, wherein the access device is free of an energy storage unit.

10. The medical system according to claim 1, wherein the measuring unit is configured to carry out an image detection of the at least one position indicator.

11. The medical system according to claim 1,
wherein the access device comprises a plurality of position indicators arranged at different positions of the access device,
and wherein the position determining unit is configured to take into account the different positions of the position indicators when determining the position of the access device.

12. The medical system according to claim 1,
wherein the robot controller is configured to generate control commands for controlling the robot with respect to a robot coordinate system,
and wherein the pivot point determining unit is configured to determine the pivot point in relation to the robot coordinate system.

13. The medical system according to claim 1,
wherein the access device has a predefined and/or predefinable target pivot point, wherein it is provided that the target pivot point substantially coincide with the pivot point during a performance of the pivot movements of the medical instrument,
and wherein the pivot point determining unit is configured to take into account a geometric relationship between the position indicator and the target pivot point when determining the pivot point for the access device.

14. The medical system according to claim 13, further comprising a user interface via which a user can predefine the target pivot point and in particular a distance of the target pivot point from the at least one position indicator.

15. A method for operating a medical system , in particular a system-assisted system for minimally invasive surgery, in particular according to the medical system set forth in claim 1, wherein the medical system comprises:
a robot configured to hold and move a medical instrument; and
an access device arrangeable to provide access to a body cavity for a medical instrument held by the robot, wherein the access device comprises at least one position indicator;
comprising the steps of:
carrying out a sensor measurement directed at the at least one position indicator;
determining the position of the access device according to the sensor measurement;
determining a pivot point for the access device according to the determined position of the access device; and
generating control commands for controlling the robot according to the determined pivot point in such a way that the robot carries out pivot movements of the medical instrument about the pivot point.

16. A program code comprising instructions that, when executed by a processor, cause a method according to claim 15 to be carried out.

17. A computer program product comprising a machine-readable medium on which program code according to claim 16 is stored.
